(19) United States
(12) Patent Application Publication
(10) Pub. No.: US 2025/0290861 A1
(43) Pub. Date: Sep. 18, 2025

(54) INSPECTION APPARATUS

(71) Applicant: ANRITSU CORPORATION, Kanagawa (JP)

(72) Inventors: Jyunichi SANO, Kanagawa (JP); Eiji TANIGUCHI, Kanagawa (JP); Tetsuro HINOKI, Kanagawa (JP)

(21) Appl. No.: 19/077,129

(22) Filed: Mar. 12, 2025

(30) Foreign Application Priority Data
Mar. 15, 2024 (JP) 2024-041542

(51) Int. Cl. G01N 21/88; G01N 33/94

(52) U.S. Cl. CPC G01N 21/8806; G01N 2021/8835; G01N 33/94; G01N 2201/06113

(57) ABSTRACT

An inspection apparatus that enables a foreign object contained in or attached to an object as an inspection target to be easily detected and that can improve accuracy of inspection is provided. An inspection apparatus that inspects an object as an inspection target at least partially including a non-transparent region allowing transmission of visible light includes a light source unit that emits the visible light to the object at an inspection position, an imaging unit that is disposed in a side opposite to the light source unit with the object interposed between the imaging unit and the light source unit and that images transmitted light transmitted through the object, and an inspection unit that inspects the object based on an image captured by the imaging unit.

### TECHNICAL FIELD

The present invention relates to an inspection apparatus.

### BACKGROUND ART

In the related art, known inspection apparatuses that inspect objects include a tablet inspection apparatus that emits an X-ray to a continuously transported PTP film from a d orthogonal to a transport direction, acquires an X-ray transmission image of the whole PTP film by performing one-dimensional sequential scanning (exposure) with the X-ray transmitted through the PTP film using, for example, imaging means such as an X-ray line camera, and performs inspection related to presence or absence, a crack, missing, and the like of a tablet based on the acquired X-ray transmission image (for example, refer to Patent Document 1).

### RELATED ART DOCUMENT

### Patent Document

[Patent Document 1] JP-A-2014-85190

### DISCLOSURE OF THE INVENTION

### Problem that the Invention is to Solve

However, in the inspection apparatus that inspects an object using light easily transmitted through the object, such as radiation including an X-ray, as disclosed in Patent Document 1, a problem arises in that, in a case where a foreign object through which the radiation or the like is transmitted is contained in or attached to the object as an inspection target, it is difficult to detect the foreign object, and accuracy of inspection is decreased.

The present invention has been conceived in view of the above circumstances, and an object thereof is to provide an inspection apparatus that enables a foreign object contained in or attached to an object as an inspection target to be easily detected and that can improve accuracy of inspection.

### Means for Solving the Problem

According to a first aspect of the present invention, there is provided an inspection apparatus that inspects an object at least partially including a non-transparent region allowing transmission of at least any one of visible light or near-infrared light, the apparatus including a light source unit that emits the visible light and/or the near-infrared light transmittable through the object, a first imaging unit that is disposed in a side opposite to the light source unit with the object interposed between the first imaging unit and the light source unit and that images transmitted light transmitted through the object, and an inspection unit that inspects the object based on an image captured by the first imaging unit.

With this configuration, in the inspection apparatus of the first aspect of the present invention, the light source unit emits at least any one of the visible light or the near-infrared light to the object that is at least partially non-transparent under a condition in which the light is not emitted, and the inspection unit inspects the object based on the image obtained by imaging the transmitted light of the object via the imaging unit. Thus, a foreign object contained in or attached to the object as an inspection target can be captured using at least any one of the visible light or the near-infrared light. Accordingly, the foreign object contained in or attached to the object as the inspection target can be easily detected, and accuracy of inspection can be improved.

The inspection apparatus according to the present invention uses the visible light or the near-infrared light in a manner that is harmless to a human body. Thus, for example, compared to use of light such as radiation affecting a human body, safety is achieved, and the apparatus can be simplified because configurations such as a shielding mechanism are not necessary.

According to a second aspect of the present invention, the inspection apparatus of the first aspect may further include a second imaging unit that images the object from a different direction from the first imaging unit, in which the inspection unit inspects the object based on respective images captured by the first imaging unit and the second imaging unit.

With this configuration, the inspection apparatus of the second aspect of the present invention includes, in addition to the first imaging unit disposed in the side opposite to the light source unit with the object interposed between the first imaging unit and the light source unit, the second imaging unit that images the object from the different direction from the first imaging unit, and the inspection unit inspects the object based on the respective images captured by the first imaging unit and the second imaging unit. Thus, inspection can be performed with reduced blind spots on the object, and the foreign object contained in or attached to the object as the inspection target can be more securely detected.

According to a third aspect of the present invention, in the inspection apparatus of the second aspect, the second imaging unit may include a plurality of imaging devices, and the plurality of imaging devices may be disposed to image the object from directions different from each other.

With this configuration, in the inspection apparatus of the third aspect of the present invention, the second imaging unit includes the plurality of imaging devices disposed to image the object from different directions. Thus, blind spots on the object can be further reduced.

According to a fourth aspect of the present invention, in the inspection apparatus of the first aspect, a light-blocking member that blocks stray light during inspection may be provided between the object and the first imaging unit. According to a fifth aspect of the present invention, in the inspection apparatus of the second aspect, a light-blocking member that blocks stray light during inspection may be provided between the object and the first imaging unit. According to a sixth aspect of the present invention, in the inspection apparatus of the third aspect, a light-blocking member that blocks stray light during inspection may be provided between the object and the first imaging unit.

With this configuration, in the inspection apparatus of the fourth to sixth aspects of the present invention, the light-blocking member that blocks the stray light is provided between the object and the imaging unit at the inspection position. Thus, light not transmitted through the object can be prevented from being incident on the imaging unit as the stray light.

According to a seventh aspect of the present invention, in the inspection apparatus of the first aspect, the light source unit may be configured to emit light not having directivity, and a diaphragm member that narrows light heading to the object from the light source unit during inspection may be provided between the object and the light source unit. According to an eighth aspect of the present invention, in the inspection apparatus of the second aspect, the light source unit may be configured to emit light not having directivity, and a diaphragm member that narrows light heading to the object from the light source unit during inspection may be provided between the object and the light source unit. According to a ninth aspect of the present invention, in the inspection apparatus of the third aspect, the light source unit may be configured to emit light not having directivity, and a diaphragm member that narrows light heading to the object from the light source unit during inspection may be provided between the object and the light source unit. According to a tenth aspect of the present invention, in the inspection apparatus of the fourth aspect, the light source unit may be configured to emit light not having directivity, and a diaphragm member that narrows light heading to the object from the light source unit during inspection may be provided between the object and the light source unit.

With this configuration, in the inspection apparatus of the seventh to tenth aspects of the present invention, the diaphragm member is provided between the object and the light source unit at the inspection position. Thus, unnecessary light other than light incident on the object in at least any one of the visible light or the near-infrared light emitted from the light source unit can be prevented from heading to a side closer to the imaging unit.

According to an eleventh aspect of the present invention, the inspection apparatus of the first aspect may further include a transport unit that moves the object by holding the object on an end surface of the object different from an side incidence surface and a transmission side surface of the object. According to a twelfth aspect of the present invention, the inspection apparatus of the second aspect may further include a transport unit that moves the object by holding the object on an end surface of the object different from an incidence side surface and a transmission side surface of the object. According to a thirteenth aspect of the present invention, the inspection apparatus of the fourth aspect may further include a transport unit that moves the object by holding the object on an end surface of the object different from an incidence side surface and a transmission side surface of the object. According to a fourteenth aspect of the present invention, the inspection apparatus of the seventh aspect may further include a transport unit that moves the object by holding the object on an end surface of the object different from an incidence side surface and a transmission side surface of the object.

With this configuration, the inspection apparatus of the eleventh to fourteenth aspects of the present invention can implement stable transport while exposing the incidence side surface to which the light from the light source unit is emitted and the transmission side surface through which the transmitted light passes. Thus, inspection can be performed with high accuracy even while the object is moving.

According to a fifteenth aspect of the present invention, in the inspection apparatus of the first aspect, the object may be a pharmaceutical formulation. According to a sixteenth aspect of the present invention, in the inspection apparatus of the second aspect, the object may be a pharmaceutical formulation. According to a seventeenth aspect of the present invention, in the inspection apparatus of the third aspect, the object may be a pharmaceutical formulation. According to an eighteenth aspect of the present invention, in the inspection apparatus of the fourth aspect, the object may be a pharmaceutical formulation. According to a nineteenth aspect of the present invention, in the inspection apparatus of the seventh aspect, the object may be a pharmaceutical formulation. According to a twentieth aspect of the present invention, in the inspection apparatus of the eleventh aspect, the object may be a pharmaceutical formulation.

With this configuration, the inspection apparatus of the fifteenth to twentieth aspects of the present invention can also be applied to a case where the object as the inspection target is, for example, a tablet of which the whole surface is normally not transparent.

### Advantage of the Invention

According to the present invention, an inspection apparatus that enables a foreign object contained in or attached to an object as an inspection target to be easily detected and that can improve accuracy of inspection can be provided.

### BRIEF DESCRIPTION OF THE DRAWINGS

FIG. 1 is a schematic configuration diagram of an inspection apparatus according to a first embodiment of the present invention.

FIGS. 2A to 2C are perspective views illustrating an example of a transport unit that transports tablets to the inspection apparatus according to the first embodiment of the present invention.

FIG. 3 is a perspective view illustrating another example of the transport unit that transports tablets to the inspection apparatus according to the first embodiment of the present invention.

FIG. 4 is a diagram illustrating an example of an image of a tablet captured by the inspection apparatus according to the first embodiment of the present invention.

FIG. 5A is an image obtained by imaging an exterior of the tablet, and FIG. 5B is an image obtained by imaging transmitted light of the tablet via the inspection apparatus according to the first embodiment of the present invention.

FIG. 6 is a diagram a illustrating modification example of the inspection apparatus according to the first embodiment of the present invention.

FIG. 7 is a diagram illustrating a modification example of a light source unit of the inspection apparatus according to the first embodiment of the present invention.

FIG. 8 is a schematic configuration diagram of an inspection apparatus according to a second embodiment of the present invention.

FIG. 9 is a diagram illustrating a modification example of the inspection apparatus according to the second embodiment of the present invention.

FIG. 10 is a diagram illustrating an example of an image of a tablet captured by a second imaging unit of the inspection apparatus according to the modification example illustrated in FIG. 9.

FIG. 11 is a diagram illustrating another example of the image of the tablet captured by the second imaging unit of the inspection apparatus according to the modification example illustrated in FIG. 9.

FIG. 12 is a top view of the modification example of the inspection apparatus according to the second embodiment of the present invention.

### BEST MODE FOR CARRYING OUT THE INVENTION

Hereinafter, an inspection apparatus according to embodiments of the present invention will be described with reference to the drawings.

### First Embodiment

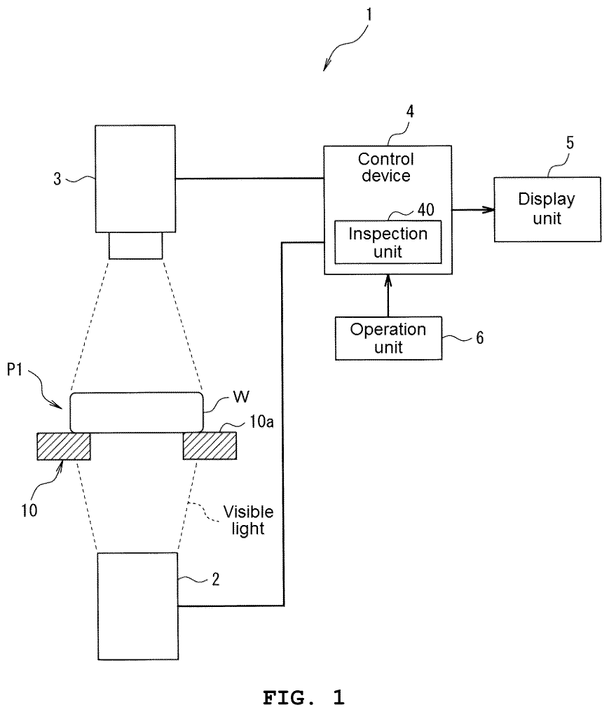

As illustrated in FIG. 1, an inspection apparatus 1 according to a first embodiment emits visible light to an object as an inspection target at a predetermined inspection position while the object is being aligned and individually transported by an existing transport unit, images transmitted light transmitted through the object by the emission of the visible light, and inspects quality of the object based on a captured image of the object. Examples of the quality of the object inspected by the inspection apparatus 1 of the present embodiment include presence or absence of a foreign object (including, for example, a liquid, a powder coating, or the like) contained in or attached to the object.

Examples of the existing transport unit include a transport unit configured to align and individually transport the object, such as a transport belt, a transport disc, or a transport chute. In the present embodiment, an example of using a transport belt 10 having a horizontal transport surface 10a (refer to FIG. 1) as the existing transport unit will be described.

The object as the inspection target is an object that includes a non-transparent region allowing transmission of the visible light as a whole or that partially includes the non-transparent region. The object as the inspection target includes not only a non-packaged individually transportable object having an outer diameter φ of a several mm to a several tens of mm and a bite-sized object but also an object or a molded object of a predetermined shape manufactured in an existing manufacturing facility or a manufacturing facility not provided with an inspection function, particularly an object not changing in shape during a transport process.

Examples of the object include a pharmaceutical formulation such as a tablet, a capsule, a troche, or a drop, and a candy. Hereinafter, a tablet W having a circular shape in a plan view and a substantially columnar shape in a side view with a height (a thickness) smaller than a diameter will be described as an example of the object as the inspection target. The object as the inspection target is not limited to a circular shape in a plan view and is applicable to objects of various shapes such as an elliptical shape and a polygonal shape.

The inspection apparatus 1 according to the present embodiment is configured to include a light source unit 2, an imaging unit 3, and a control device 4.

The inspection apparatus 1 according to present embodiment emits the visible light to the tablet W being transported by the transport belt 10 at a predetermined inspection position P1, images transmitted light transmitted through the tablet W by the emission of the visible light, and inspects the quality of the tablet W based on a captured image of the tablet W.

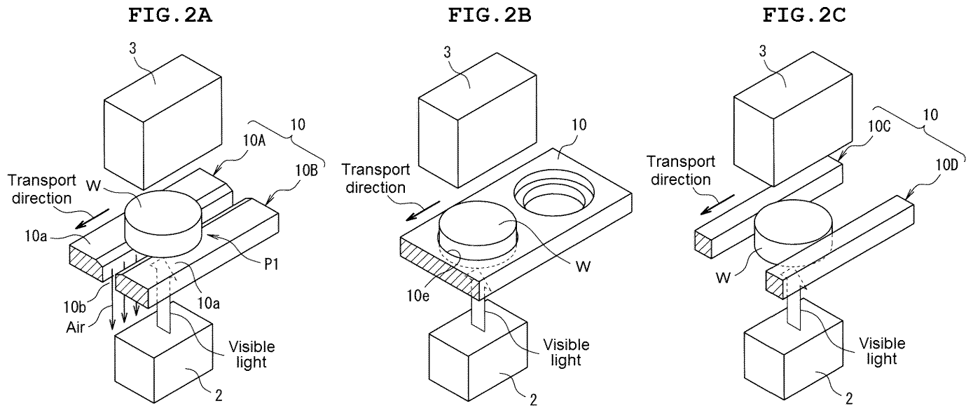

As illustrated in FIG. 2A, the transport belt 10 is a transport belt that consists of, for example, a first transport belt 10A and a second transport belt 10B arranged parallel to each other with a predetermined gap 10b between the first transport belt 10A and the second transport belt 10B and that is configured to transport the tablet W using air to hold a lower surface of the tablet W on the transport surface 10a through the predetermined gap 10b.

The transport surface 10a is configured with an upper surface of the first transport belt 10A and an upper surface of the second transport belt 10B. In FIG. 1, the transport surface 10a of the transport belt 10 is used as a transport path of the tablet W, and the transport path extends in a direction orthogonal to the page of FIG. 1.

An existing transport belt for transporting the tablet W by holding the lower surface of the tablet W on the transport surface as in the present embodiment may be, for example, as illustrated in FIG. 2B, a transport belt that is configured with a single transport belt 10 and that has a transport surface on which a placement hole 10e is formed. The placement hole 10e has a stepped shape, and the tablet W fits in the step. This configuration may be further stabilized using air suction.

As illustrated in FIG. 2C, the transport belt 10 may be in the form of transporting the tablet W by holding the tablet W between a transport belt 10C and a transport belt 10D.

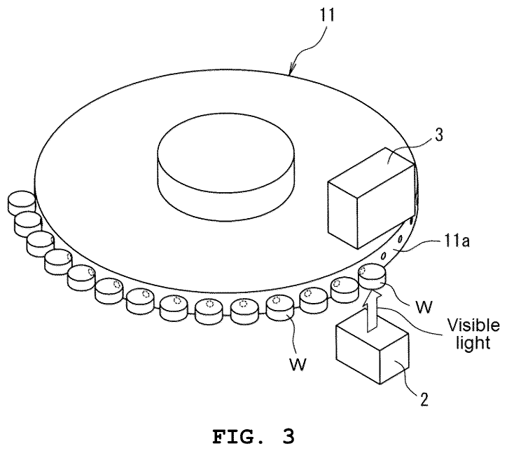

In a case where a transport disc is used as the existing transport unit, a transport disc 11, for example, as illustrated in FIG. 3, can be used. As illustrated in FIG. 3, the transport disc 11 is a transport unit configured to transport the tablet W in a circumferential direction while laterally rotating with the tablet W held in a suction hole of an outer circumferential surface 11a by suction. In the transport disc 11, the tablet W is transported in a state where an upper surface (referred to as a transmission side surface) and the lower surface (referred to as an incidence side surface) of the tablet W are horizontally maintained by holding a side surface of the tablet W in the suction hole by suction. In a case where the transport disc 11 is used, a plane orthogonal to the outer circumferential surface 11a, that is, a plane on which the lower surface of the tablet W moves, is defined as the transport surface.

### Light Source Unit

As illustrated in FIG. 1, the light source unit 2 is disposed in a side opposite to the imaging unit 3 (in the present embodiment, in a side below the transport belt 10) with the tablet W and the transport belt 10 at the inspection position P1 interposed between the light source unit 2 and the imaging unit 3, and emits the visible light to the tablet W at the inspection position P1. Specifically, the light source unit 2 emits the visible light to a surface (in the present embodiment, the lower surface) of the tablet W on a side opposite to a side closer to the imaging unit 3.

The light source unit 2 is held by a bracket (not illustrated) at a position separated by a predetermined distance from the transport belt 10.

Any light source capable of emitting the visible light may be used as the light source unit 2. For example, various light sources such as a broadband light source represented by a halogen lamp, and a visible ray LED light can be used.

The light source unit 2 is configured to adjust a light quantity of the visible light to be emitted. For example, the light quantity of the light source unit 2 may be adjusted automatically by the control device 4 or by an operation of a user through an operation unit 6, described later.

The light source unit 2 may be configured to change a wavelength of the visible ray in accordance with, for example, characteristics of the inspection target or defects anticipated in the inspection target.

### Imaging Unit

The imaging unit 3 is disposed in a side opposite to the light source unit 2 (in the present embodiment, in a side above the transport belt 10) with the tablet W and the transport belt 10 at the inspection position P1 interposed between the imaging unit 3 and the light source unit 2, and images the transmitted light that is emitted from the light source unit 2 and transmitted through the tablet W at the inspection position P1.

The imaging unit 3 is held by a bracket (not illustrated) to have an optical axis orthogonal to the transport surface 10a from a position separated by a predetermined height from the transport surface 10a of the transport belt 10.

The imaging unit 3 consists of an area camera using a two-dimensional which pixels are two-dimensionally arranged on a light-receiving surface as an imaging element. A line camera using a one-dimensional element in which pixels are one-dimensionally arranged on a light-receiving surface as an imaging element may be used as the imaging unit 3.

The imaging unit 3 is configured to adjust an imaging time, that is, an exposure time, for the tablet W. For example, the exposure time of the imaging unit 3 may be adjusted automatically by the control device 4 or by an operation of the user through the operation unit 6, described later.

### Control Device

The control device 4 includes a CPU and a memory or the like as a storage region or a work region of a control program and controls the whole inspection apparatus 1. Content of controls of the control device 4 includes not only a control of display content and a display aspect of a display unit 5 but also, for example, a control for performing various requests or settings corresponding to an operation input through the operation unit 6.

The display unit 5 and the operation unit 6 are connected to the control device 4.

The display unit 5 is configured with a flat display or the like and performs display output to the user. The display unit 5 displays an image of an inspection result or the like provided by the control device 4.

For example, the display unit 5 may display the inspection result of the tablet W using a text such as “OK” and “NG” or a symbol or may display a statistical value such as a total number of inspections, the number of normal objects, and a total number of NGs.

The display unit 5 may display an image of the tablet W captured by the imaging unit 3. In this case, the user can visually inspect the quality based on the image of the tablet W displayed on the display unit 5.

The display content and the display aspect of the display unit 5 are determined based on, for example, a default setting or a request made by a predetermined key operation from the operation unit 6.

The operation unit 6 consists of, for example, a touch panel display and is operated by the user to perform various settings and various types of display. The operation unit 6 and the display unit 5 may be configured to be integrated as a touch panel display.

For example, the control device 4, the display unit 5, and the operation unit 6 may be configured to be integrated with the light source unit 2 or the imaging unit 3 as a unit or may be configured with a terminal (for example, a personal computer or a portable terminal) provided separately from the light source unit 2 or the imaging unit 3.

The control device 4 includes an inspection unit 40 that inspects the tablet W based on the image of the tablet W captured by the imaging unit 3.

The inspection unit 40 specifies a region of the tablet W in the image by performing, for example, image processing such as binarization or edge detection on the image of the tablet W captured by the imaging unit 3. The inspection unit 40 determines whether or not any foreign object through which, for example, radiation or an infrared ray is transmitted is contained in or attached to the tablet W in the above specified region showing the tablet W in the image. For example, the inspection unit 40 can determine whether or not the foreign object is contained in or attached to the tablet W by detecting a defective part from the image of the tablet W captured by the imaging unit 3 and determining whether or not the detected defective part is present in the region of the tablet W.

### Example of Captured Image

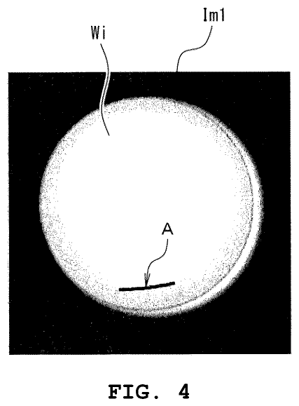

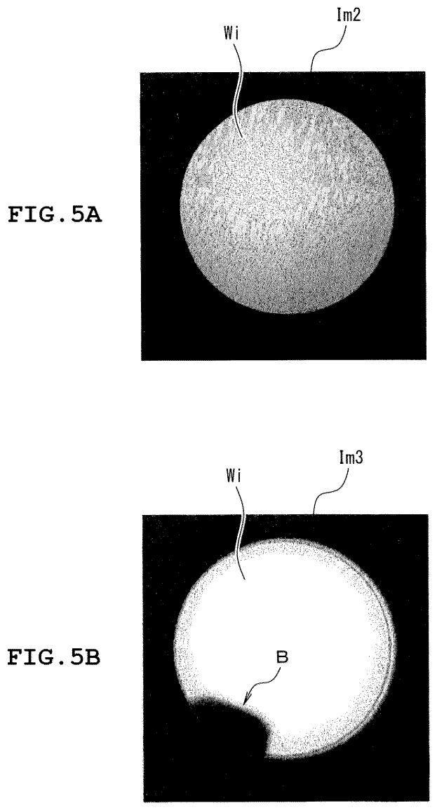

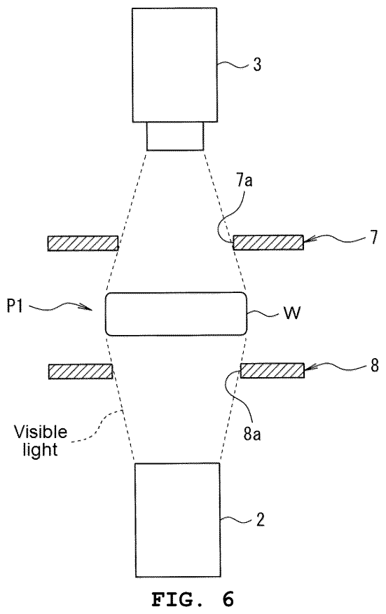

Next, the image of the tablet W captured by the imaging unit 3 of present embodiment will be described using the images illustrated in FIGS. 4 and 5A and 5B as examples.

FIG. 4 is an example of the image in a case where the tablet W to which a hair is attached on a surface (in the present embodiment, the upper surface) is imaged.

As in an image Im1 illustrated in FIG. 4, the tablet W is imaged in a state where an inner part of the tablet W is illuminated by the visible light emitted from the light source unit 2. In FIG. 4, a part denoted by Wi is an image showing the region of the tablet W in the image Im1.

As illustrated in FIG. 4, by illuminating the tablet W, the hair (indicated by arrow A in FIG. 4) having different transmittance of the visible light from a tablet part is captured as a shadow. Accordingly, the fact that the hair is contained in or attached to the tablet W is easily determined based on the image Im1.

In the present embodiment, the visible light is also transmitted through a peripheral part of the tablet W. Thus, the peripheral part of the tablet W is also brightly illuminated and not shadowed, as in the image Im1. Accordingly, for example, even in a case where the foreign object such as the hair is present in the peripheral part of the tablet W, the foreign object can be easily detected.

FIGS. 5A and 5B are examples of the image in a case where the tablet W to which an unusual coating is attached on a surface (in the present embodiment, the upper surface) is imaged.

The tablet W to which the unusual coating is attached will be described by comparing the image showing an exterior in FIG. 5A with the image obtained by imaging the transmitted light in FIG. 5B. In FIGS. 5A and 5B, the part denoted by Wi is an image showing the region of the tablet W in each image of an image Im2 and an image Im3.

As illustrated in FIG. 5A, it may be difficult to determine whether or not the unusual coating is attached to the tablet W from the image obtained by imaging the exterior of the tablet W, depending on a type of the unusual coating attached to the tablet W.

In the present embodiment, even in a case where the unusual coating for which it is difficult to determine its presence or absence from the exterior as described above is attached to the tablet W, attachment of the unusual coating can be easily detected by obtaining the image Im3 in a state where the inner part is illuminated by the visible light emitted from the light source unit 2, as illustrated in FIG. 5B. In the image Im3, the unusual coating (indicated by arrow B in FIG. 5B) having different transmittance of the visible light from the tablet part is captured as a shadow.

### Operational Effects

As described above, in the inspection apparatus according to the present embodiment, the light source unit 2 emits the visible light to the tablet W as the inspection target, and the inspection unit 40 inspects the tablet W based on the image obtained by imaging the transmitted light of the tablet W via the imaging unit 3. Thus, the foreign object contained in or attached to the tablet W as the inspection target can be captured using the visible light. Accordingly, the foreign object contained in or attached to the tablet W as the inspection target can be easily detected, and accuracy of inspection can be improved.

The inspection apparatus according to the present embodiment uses the visible light in a manner that is harmless to a human body. Thus, for example, compared to use of light such as radiation affecting a human body, safety is achieved, and the apparatus can be simplified because configurations such as a shielding mechanism are not necessary.

### Modification Example

In the present embodiment, as illustrated in FIG. 6, a light-blocking member 7 that blocks stray light may be provided between the tablet W and the imaging unit 3 at the inspection position P1. An opening 7a having a circular shape corresponding to a shape of the tablet W is formed in the light-blocking member 7. An opening area and a shape of the opening 7a are appropriately set in accordance with a size and a shape of the inspection target.

Ideally, the light-blocking member 7 allows only the transmitted light transmitted through the tablet W in the visible light emitted from the light source unit 2 to pass through the opening 7a, thereby preventing light not transmitted through the tablet W from being incident on the imaging unit 3 as stray light.

For example, the light-blocking member 7 may be supported by the bracket holding the imaging unit 3, may be supported by a lens barrel of the imaging unit 3, or may be supported by other supporting means.

In the present embodiment, a diaphragm member 8 that narrows the visible light heading to the tablet W from the light source unit 2 may be provided between the tablet W and the light source unit 2 at the inspection position P1.

A light-blocking plate in which an opening 8a having a circular shape corresponding to the shape of the tablet W is formed can be used as the diaphragm member 8. The diaphragm member 8 is not limited to a light-blocking plate, and any member capable of narrowing the visible light heading to the tablet W may be used. For example, an iris or a cylindrical member having a tapered shape having a diameter gradually reduced in a direction of a side closer to the tablet W may be used. A condensing lens may be used as the diaphragm member 8.

The diaphragm member 8 prevents unnecessary light other than light incident on the tablet W in the visible light emitted from the light source unit 2 from heading to a side closer to the imaging unit 3. The diaphragm member 8 is effective in a case where the visible light emitted from the light source unit 2 is light having low directivity.

For example, the diaphragm member 8 may be supported by the bracket holding the light source unit 2, may be supported by the light source unit 2, or may be supported by other supporting means.

Both of the light-blocking member 7 and the diaphragm member 8 described above may be provided as illustrated in FIG. 6, or any one of the light-blocking member 7 or the diaphragm member 8 may be configured to be provided.

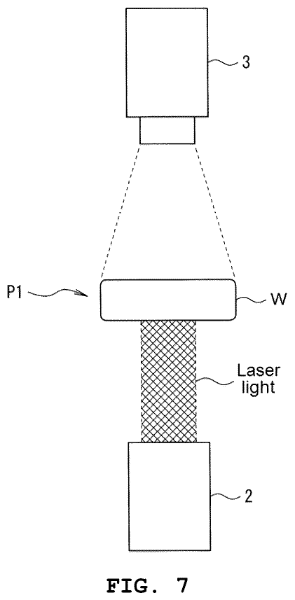

In the present embodiment, as illustrated in FIG. 7, a laser light source capable of emitting laser light having high directivity may be used as the light source unit 2. In this case, the visible light emitted to the tablet W is light having high directivity. Thus, need for both or one of the light-blocking member 7 or the diaphragm member 8 described above can be eliminated.

While the light source unit 2 is configured to emit the visible light in the present embodiment described above, the present invention is not limited to this, and the light source unit 2 may be configured to emit near-infrared light, or the visible light and the near-infrared light. In a case where the light source unit 2 is configured to emit the near-infrared light, the object as the inspection target is preferably an object that includes a non-transparent region allowing transmission of the near-infrared light as a whole or that partially includes the non-transparent region. In a case where the light source unit 2 is configured to emit the visible light and the near-infrared light, the object as the inspection target is preferably an object that includes a non-transparent region allowing transmission of the visible light or the near-infrared light as a whole or that partially includes the non-transparent region.

### Second Embodiment

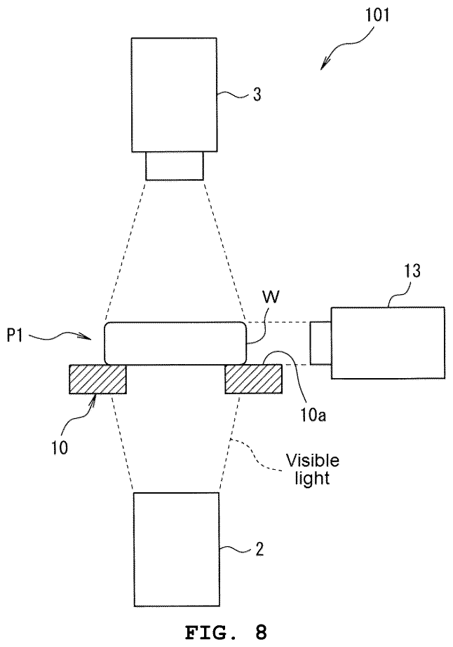

An inspection apparatus 101 according to a second embodiment of the present invention will be described with reference to FIGS. 8 to 10.

The present embodiment has a different number of imaging units from the first embodiment, but other configurations are the same as those in the first embodiment. Accordingly, the same configurations as the first embodiment will not be described.

As illustrated in FIG. 8, the inspection apparatus 101 according to the present embodiment further includes, in addition to the imaging unit 3, a second imaging unit 13 that images the tablet W from a direction (in the present embodiment, a lateral direction of the tablet W) different from that of the imaging unit 3, as a configuration for imaging the tablet W. The second imaging unit 13 consists of an area camera, like the imaging unit 3. A line camera may be used as the second imaging unit 13.

The second imaging unit 13 is disposed in the lateral side of the tablet W at the inspection position P1 and images the tablet W from the lateral side. The second imaging unit 13 is connected to the control device 4, like the imaging unit 3. Thus, the control device 4 can obtain an image of a view of the tablet N from the side in addition to the image of the tablet W captured by the imaging unit 3. The inspection unit 40 inspects the tablet W based on the image of the tablet W captured by the imaging unit 3 and on the image of the tablet W captured by the second imaging unit 13.

### Operational Effects

As described above, the inspection apparatus according to the present embodiment includes, in addition to the imaging unit 3 disposed in the side opposite to the light source unit 2 with the tablet W interposed between the imaging unit 3 and the light source unit 2, the second imaging unit 13 that images the tablet W from the different direction from the imaging unit 3, and the inspection unit 40 inspects the tablet W based on the respective images captured by the imaging unit 3 and the second imaging unit 13. Thus, inspection can be performed with reduced blind spots on the tablet W, and the foreign object contained in or attached to the tablet W as the inspection target can be more securely detected.

### Modification Example

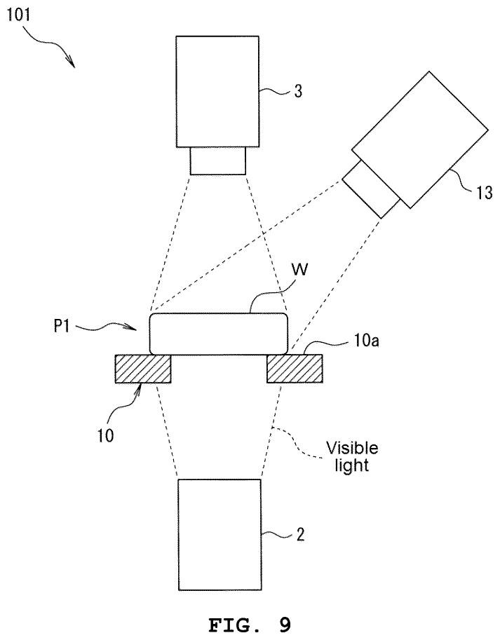

In the present embodiment, as illustrated in FIG. 9, the second imaging unit 13 may be disposed in an oblique upward direction of the tablet W and image the tablet W from the oblique upward direction of the tablet W as the different direction from the imaging unit 3.

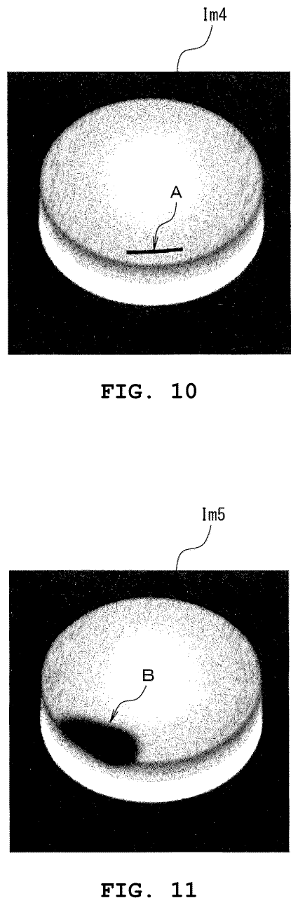

In a case where the second imaging unit 13 is disposed in the oblique upward direction of the tablet W, the image of the tablet W captured by the second imaging unit 13 is obtained as, for example, the image illustrated in FIG. 10 or 11.

FIG. 10 is a diagram illustrating an image Im4 obtained by imaging the same tablet W as FIG. 4 described in the first embodiment via the second imaging unit 13 illustrated in FIG. 9. FIG. 11 is a diagram illustrating an image Im5 obtained by imaging the same tablet W as FIG. 5B described in the first embodiment via the second imaging unit 13 illustrated in FIG. 9.

Even in the present embodiment, the hair (indicated by arrow A in FIG. 10) is captured as a shadow in the image Im4, as illustrated in FIG. 10. In the image Im4, the fact that the hair is positioned on an upper surface side of the tablet W can also be identified.

In the image Im5, the unusual coating (indicated by arrow B in FIG. 11) attached to the tablet W is captured as a shadow, as illustrated in FIG. 11. In the image Im5, the fact that the unusual coating is positioned on the upper surface side of the tablet W can also be identified.

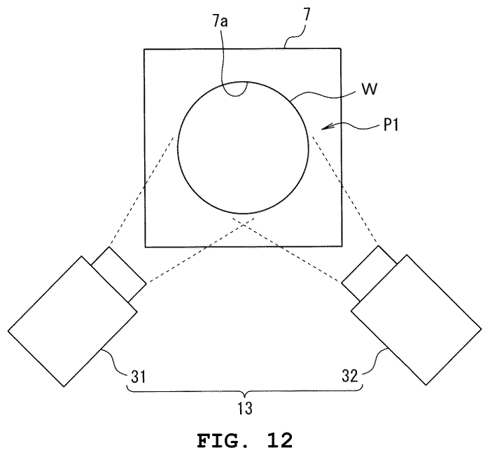

While the second imaging unit 13 is configured to consist of one imaging device such as an area camera in the present embodiment, the second imaging unit 13 may be configured to consist of a plurality of imaging devices (in the example illustrated in FIG. 12, two), as illustrated in FIG. 12. The number of imaging devices is not limited to two and may be three or more.

As illustrated in FIG. 12, the second imaging unit 13 includes an imaging device 31 and an imaging device 32, and the imaging device 31 and the imaging device 32 are disposed to image the tablet W from directions different from each other.

The imaging device 31 and the imaging device 32 may be disposed in an upstream side or a downstream side of the tablet W at the inspection position P1 in a transport direction of the tablet W. The imaging device 31 and the imaging device 32 may be disposed in a direction orthogonal to the transport direction of the tablet W. The imaging device 31 and the imaging device 32 may be disposed at any positions at which the tablet W can be imaged from directions different from each other in a top view, and are preferably appropriately disposed at optimal positions in accordance with specifications of the inspection apparatus 101 or a type of the inspection target.

In the example illustrated in FIG. 12, the second imaging unit 13 includes the imaging devices 31 and 32 disposed to image the tablet W from different directions. Thus, blind spots on the tablet W can be further reduced. While an example in which the light-blocking member 7 is provided is disclosed in FIG. 12, the light-blocking member 7 may not be provided.

The imaging device 31 and the imaging device 32 may be disposed to image the tablet W from directions different from each other even in an up-down direction. For example, the imaging device 31 may be disposed in the lateral side of the tablet Was illustrated in FIG. 8, and the imaging device 32 may be disposed in the oblique upward direction of the tablet Was illustrated in FIG. 9.

While embodiments of the present invention have been disclosed, changes may be made by those skilled in the art without departing from the scope of the present invention. All of such corrections and equivalents are intended to fall within the following claims.

### DESCRIPTION OF REFERENCE NUMERALS AND SIGNS

1, 101: inspection apparatus
2: light source unit
3: imaging unit (first imaging unit)
4: control device
5: display unit
6: operation unit
7: light-blocking member
7a: opening
8: diaphragm member
8a: opening
10: transport belt
11: transport disc (transport unit)
13: second imaging unit
31, 32: imaging device
40: inspection unit
W: tablet (object)
P1: inspection position
Im1, Im2, Im3, Im4, Im5: image

What is claimed is:

1. An inspection apparatus that inspects an object at least partially including a non-transparent region allowing transmission of at least any one of visible light or near-infrared light, the apparatus comprising:
a light source unit that emits the visible light and/or the near-infrared light transmittable through the object;
a first imaging unit that is disposed in a side opposite to the light source unit with the object interposed between the first imaging unit and the light source unit and that images transmitted light transmitted through the object; and
an inspection unit that inspects the object based on an image captured by the first imaging unit.

2. The inspection apparatus according to claim 1 further comprising:
a second imaging unit that images the object from a different direction from the first imaging unit,
wherein the inspection unit inspects the object based on respective images captured by the first imaging unit and the second imaging unit.

3. The inspection apparatus according to claim 2,
wherein the second imaging unit includes a plurality of imaging devices, and
the plurality of imaging devices are disposed to image the object from directions different from each other.

4. The inspection apparatus according to claim 1,
wherein a light-blocking member that blocks stray light during inspection is provided between the object and the first imaging unit.

5. The inspection apparatus according to claim 2,
wherein a light-blocking member that blocks stray light during inspection is provided between the object and the first imaging unit.

6. The inspection apparatus according to claim 3,
wherein a light-blocking member that blocks stray light during inspection is provided between the object and the first imaging unit.

7. The inspection apparatus according to claim 1,
wherein the light source unit is configured to emit light not having directivity, and
a diaphragm member that narrows light heading to the object from the light source unit during inspection is provided between the object and the light source unit.

8. The inspection apparatus according to claim 2,
wherein the light source unit is configured to emit light not having directivity, and
a diaphragm member that narrows light heading to the object from the light source unit during inspection is provided between the object and the light source unit.

9. The inspection apparatus according to claim 3,
wherein the light source unit is configured to emit light not having directivity, and
a diaphragm member that narrows light heading to the object from the light source unit during inspection is provided between the object and the light source unit.

10. The inspection apparatus according to claim 4,
wherein the light source unit is configured to emit light not having directivity, and
a diaphragm member that narrows light heading to the object from the light source unit during inspection is provided between the object and the light source unit.

11. The inspection apparatus according to claim 1, further comprising:
a transport unit that moves the object by holding the object on an end surface of the object different from an incidence side surface and a transmission side surface of the object.

12. The inspection apparatus according to claim 2, further comprising:
a transport unit that moves the object by holding the object on an end surface of the object different from an incidence side surface and a transmission side surface of the object.

13. The inspection apparatus according to claim 4 further comprising:
a transport unit that moves the object by holding the object on an end surface of the object different from an incidence side surface and a transmission side surface of the object.

14. The inspection apparatus according to claim 7, further comprising:
a transport unit that moves the object by holding the object on an end surface of the object different from an incidence side surface and a transmission side surface of the object.

15. The inspection apparatus according to claim 1,
wherein the object is a pharmaceutical formulation.

16. The inspection apparatus according to claim 2,
wherein the object is a pharmaceutical formulation.

17. The inspection apparatus according to claim 3,
wherein the object is a pharmaceutical formulation.

18. The inspection apparatus according to claim 4,
wherein the object is a pharmaceutical formulation.

19. The inspection apparatus according to claim 7, wherein the object is a pharmaceutical formulation.

20. The inspection apparatus according to claim 11,
wherein the object is a pharmaceutical formulation.
